(19) United States
(12) Patent Application Publication
(10) Pub. No.: US 2009/0252673 A1
(43) Pub. Date: Oct. 8, 2009

(54) Methods of Making and Using Rubidium-81-Containing Compositions

(76) Inventor: Hans Hendrik Van Rooij, Amsterdam (NL)

Correspondence Address: Mallinckrodt Inc., 675 McDonnell Boulevard, HAZELWOOD, MO, 63042, US

(21) Appl. No.: 12/305,260

(22) PCT Filed: Jun. 19, 2007
(86) PCT No.: PCT/US07/14308

§ 371 Date: Dec. 17, 2008

(30) Foreign Application Priority Data
Jun. 20, 2006 (GB) 0612234.5

(51) Int. Cl. A61K 51/00; B01D 59/26

(52) U.S. Cl. 424/1.11; 95/93

(57) ABSTRACT

The present invention relates to preparation of imaging compositions containing Rubidium-81. In addition, the present invention relates to uses of Rubidium-81-containing compositions in diagnostic imaging such as, for example, myocardial imaging.

This application relates to production and use of the radioisotope Rubidium-81 (“Rb-81”) for diagnostic imaging, especially by positron emission tomography (PET) or single photon emission computed tomography (SPECT). It is particularly applicable to the diagnostic imaging of cardiac tissue, for example the myocardium.

It is known that potassium channels in the myocardium may be compromised in cardiac disease. The current diagnostic agents for this are metabolic markers, perfusion markers, or agents that interfere with a sodium/potassium ATP-ase transporter. Other clearly-defined types of potassium channels are known and rubidium, a cation substitute for potassium, can image some of them and provide an alternative tool for assessing cardiac function. Until now the radioisotope Rubidium-82 (“Rb-82”) has been used in PET imaging for cardiac tissue. However, this isotope has a very short half life (78 seconds) and so has to be obtained at the site where it is to be used by eluting a Strontium/Rubidium generator. Rb-81 on the other hand has a relatively long-half life (4.7 hours) and so can be delivered to customers in what may be characterized by some as a “ready-for use” form without requiring an on-site generator and a complicated automated infusion system as is required with Rb-82.

In one aspect the invention provides a new method for the preparation of Rb-81 in a highly-pure form, in which it is suitable for incorporation into a diagnostic imaging composition that can safely be administered to a patient who is to undergo an imaging procedure such as, for example, a PET or SPECT imaging procedure.

Rb-81 having a half life of 4.7 hours is currently produced as an intermediate in the in situ preparation of gaseous Krypton-81m (Kr-81m) having a half life of 13 seconds that is clinically used for lung perfusion studies after inhalation. The method used for preparing Kr-81m is as follows:

Enriched Krypton-82 (Kr-82) is irradiated in a cyclotron, and this process generates solid Rb-81 and some impurities. The resulting Rb-81 is obtained an as aqueous solution by washing the product of the preceding step with water. Although this washing with water may remove some impurities, certain radioactive non-Rb impurities such as radioactive Br, Mn and/or Co may remain in the Rb-81 solution.

The aqueous Rb-81 solution is then absorbed onto a cation-exchange resin such that the Rb-81 becomes bound to the resin as a result of its binding affinity to negatively-charged sulfonic groups of the resin. Impurities such as those mentioned above are retained on the resin from which they can be washed with water. The resin used is generally an organic resin such as a polymer of divinylbenzene-styrene or ethylvinylbenzene.

The cation-exchange resin is then washed with water and placed in an appropriate radioisotope generator, such as a KryptoScan™ generator. The generator is then purged with air so that the gas discharged from the generator will include radioactive Kr-81m gas which is a natural decay product of Rb-81. This air including gaseous Kr-81m may then be administered to a patient by inhalation and so used for respiratory imaging by gamma scintigraphy.

We have now discovered that if the ion exchange resin to which the Rb-81 is bound is washed with a cationic solution rather than purged with air as in the production of Kr-81m gas, Rb-81 is released from the resin. Provided that the cationic solution is pharmaceutically acceptable, the Rb-81 containing solution eluted from the resin may then directly administered to a patient for diagnostic imaging using known techniques (e.g., PET and/or SPECT), after having been diluted with a pharmaceutically-acceptable diluent such as saline (0.9% NaCl) if necessary, and then sterilised.

Thus, this invention provides a procedure for obtaining a solution of Rb-81 which can be used as a pharmaceutically-acceptable imaging composition as a result of removal of the undesirable radioactive impurities that were bound to the cation-exchange resin.

According to one aspect of the invention, we therefore provide a method of preparing an imaging composition comprising a solution containing Rb-81 free from radioactive non-Rb impurities. The method includes eluting a cation-exchange material to which is bound Rb-81 with a cationic solution so as to release Rb-81 from the resin.

The radioactive non-Rb impurities may comprise one or more of radioactive bromine, radioactive manganese and radioactive cobalt

The pharmaceutically-acceptable cationic solution may, for example, be an isotonic saline solution (herein called “saline”).

The cation-exchange material may be a resin comprising beads, membrane including beads, or membrane. Suitable cation-exchange resins are, for example, polymers of ethylvinylbenzene or divinylbenzene/styrene. Examples of suitable resins are available under the brand names Dowex (e.g. Monosphere Marathon beads), PRP-X 800 and Omnipac PCX.

According to another aspect of the invention, we provide an imaging composition comprising a pharmaceutically-acceptable solution containing Rb-81 free from radioactive non-Rb impurities such as radioactive bromine, manganese, cobalt in a pharmaceutically-acceptable cationic solution.

According to a further aspect of the invention, we also provide a composition comprising a quantity of Rb-81 retained on a cation-exchange material. In order to obtain the imaging composition comprising a pharmaceutically-acceptable solution containing Rb-81 free from the radioactive non-Rb impurities, the cation-exchange material to which is bound Rb-81 can be washed with water to remove the radioactive non-Rb impurities, followed by elution of the resultant cation-exchange mixture with a pharmaceutically-acceptable cationic solution, wherein the eluting releases Rb-81 from the cation-exchange material. The cation-exchange material to which the Rb-81 is bound may be any of those described above.

According to yet another aspect of the invention, we provide use in diagnostic imaging by PET or SPECT of an imaging composition comprising a pharmaceutically-acceptable cationic solution containing Rb-81 substantially free from radioactive non-Rb impurities.

According to a further aspect of the invention, we provide a method for the preparation of an imaging composition comprising a pharmaceutically-acceptable solution of Rb-81. In this method, enriched Kr-82 gas is introduced into a cyclotron and irradiated within the cyclotron so as to generate solid Rb-81 and radioactive non-Rb impurities. The resulting product is washed with water to obtain an Rb-81-containing aqueous solution generally including at least some of the radioactive non-Rb impurities. The resulting Rb-81 containing aqueous solution is then loaded onto a cation-exchange resin, and the cationic-exchange resin is subsequently washed with water to remove remaining radioactive non-Rb impurities from the column. Later, the cation-exchange resin is eluted with a physiologically-acceptable cationic solution so as to dissociate Rb-81 from the resin. The resulting aqueous Rb-81 solution is then collected. If desired, the resulting aqueous Rb-81 solution may be diluted with a pharmaceutically-acceptable diluent. Further, the resulting aqueous Rb-81 solution and/or the diluted version thereof may be sterilised.

The above-described method of the invention may be carried out using a KryptoScan™ generator otherwise used for the production of Kr-81m as described above. The KryptoScan™ generator is loaded with a cation-exchange resin, and when that resin has absorbed onto it Rb-81 as outlined above, Kr-81m can be obtained by purging the generator with air. As the half life of Kr-81m is only 13 seconds, it must be generated immediately before it is to be inhaled by the patient so that the generator must be located at the site where the imaging is to be carried out.

When used in the present invention, however, the KryptoScan™ generator containing bound Rb-81 is eluted with a pharmaceutically-acceptable cationic solution such as isotonic saline instead of being purged by air, allowing the preparation of a physiologically-acceptable solution containing Rb-81 that after autoclaving may be administered to a patient in the PET or SPECT investigation of myocardial irregularities. On the other hand, as Rb-81 has a significantly-longer half life than Rb-82 (4.7 hours as opposed to 1.3 minutes), it is not necessary for the Rb-81 to be generated at the site of ultimate use. As an alternative it can be produced centrally and delivered to the ultimate user as a ready-to-use solution. It is therefore feasible to generate the Rb-81m in a relatively large-scale plant as well as a small scale generator such as a KryptoScan™ generator. The sterilised solution can be administered intravenously whereupon the Rb-81 will image the myocardium in the same way as Rb-82.

In a still further aspect of the invention we provide a method of performing a radioisotope elution procedure, the method comprising:
(i) eluting a cationic-exchange resin having radioactive Rb-81 thereon with a physiologically-acceptable gas so as to provide a gas mixture including the gas and Kr-81m formed by decay of the radioactive Rb-81; and
(ii) subsequently eluting the cationic-exchange resin still having at least some of the radioactive Rb-81 thereon with a physiologically-acceptable cationic solution so as to dissociate the radioactive Rb-81 from the resin.

In yet a further aspect of the invention we provide a method of obtaining air containing Kr-81m for pulmonary imaging and a pharmaceutically-acceptable solution containing Rb-81 for cardiac imaging successively from a radioisotope generator, wherein the radioisotope generator comprises a housing having a first side and a second side and a cation-exchange material located therebetween, wherein three conduits communicate with the first side of the housing and one conduit communicates with the second side, whereby
(a) in order to generate Kr-81m gas, the conduit communicating with the second side of the housing and one of the conduits communicating with the first side of the housing are closed and the remaining two conduits communicating with the first side of the housing are open, and air is supplied to the housing through one of the open conduits so that it contacts the cation exchange material, collects Kr-81m formed by decay of the Rb-81, and leaves through the other open conduit, thus providing air charged with Kr-81m suitable for pulmonary imaging; then:
(b) in order to generate the Rb-81 containing solution, the two conduits that were open are closed, and the two conduits that were closed are opened, and a physiologically-acceptable cationic solution is introduced into the housing via the open conduit communicating with the first side of the housing so that said solution passes through the cation exchange membrane and elutes Rb-81 from the membrane and the resulting Rb-81-containing solution leaves the housing through the conduit communicating with the second side thereof.

Embodiments of the invention will now be described by way of Example, and with reference to the accompanying drawings, in which:—

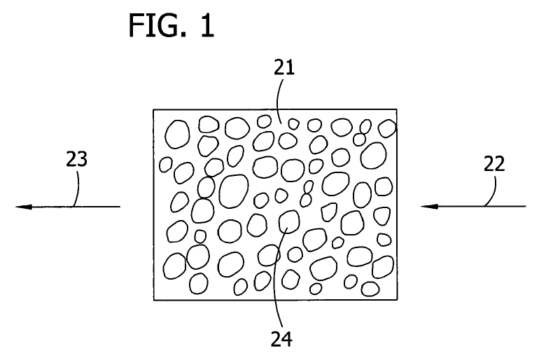

FIG. 1 is a simple block diagram showing elution of a cation exchange resin loaded with Rb-81, and

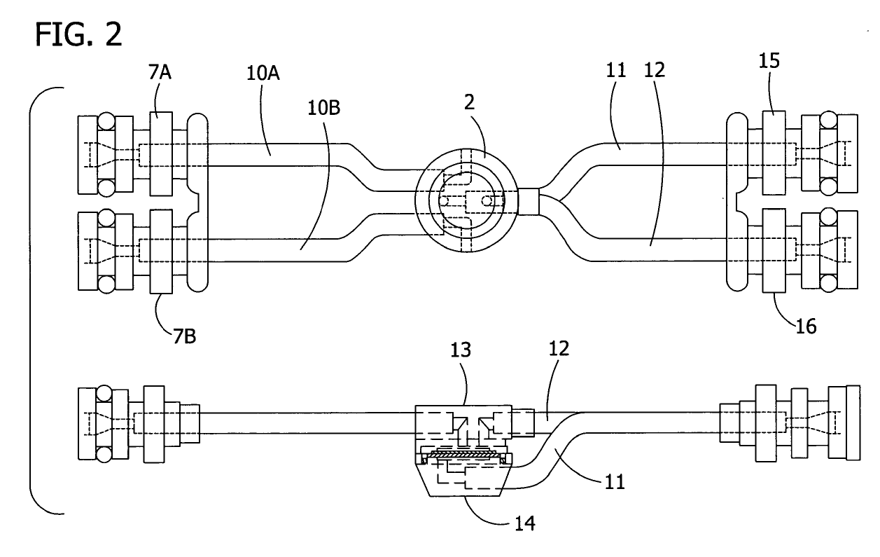

FIG. 2 illustrates the use of a KryptoScan™ generator in the production of an Rb-81-containing solution.

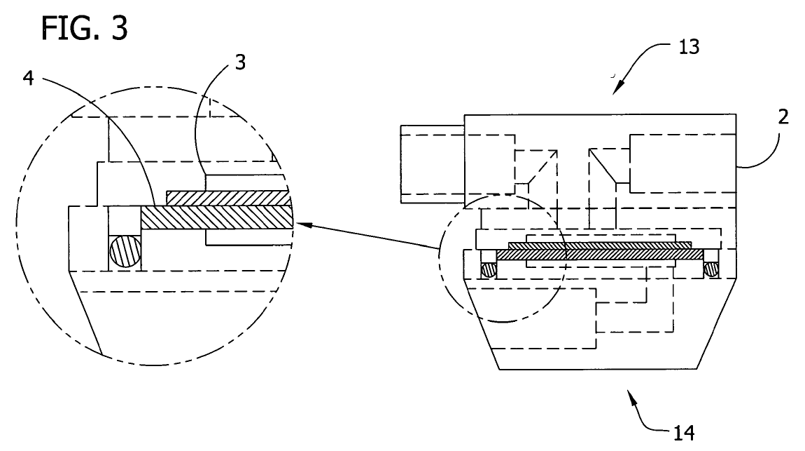

FIG. 3 shows in two stages enlarged portions of part of the generator shown in FIG. 1.

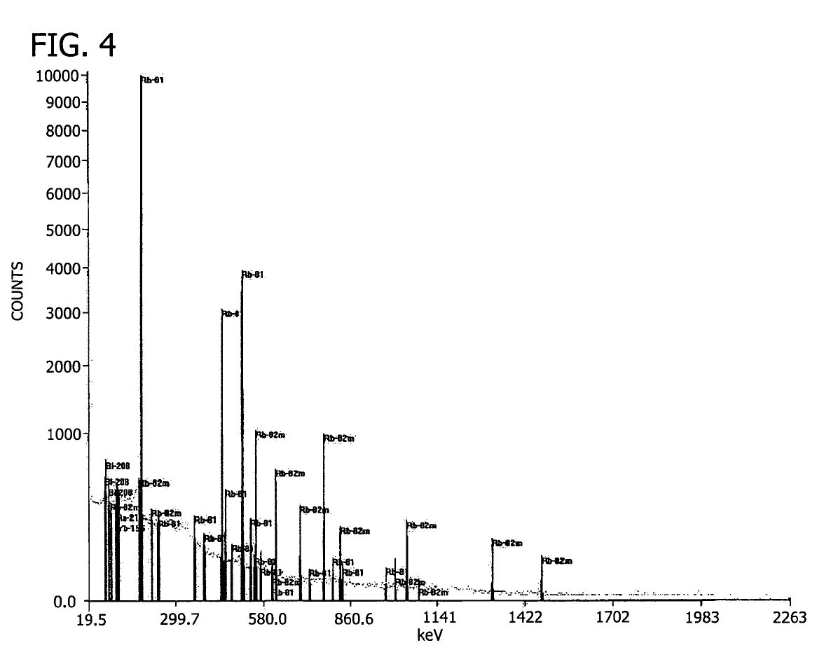

FIG. 4 shows a spectrum of Fraction 5 of generator 1 on Oct. 4, 2005.

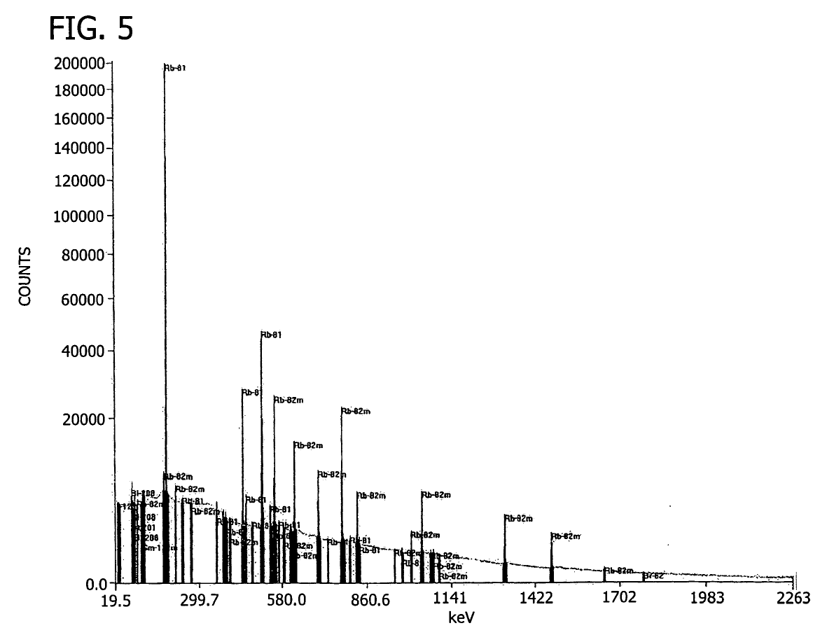

FIG. 5 shows a spectrum of Fraction 1 of generator 1 on Oct. 5, 2005.

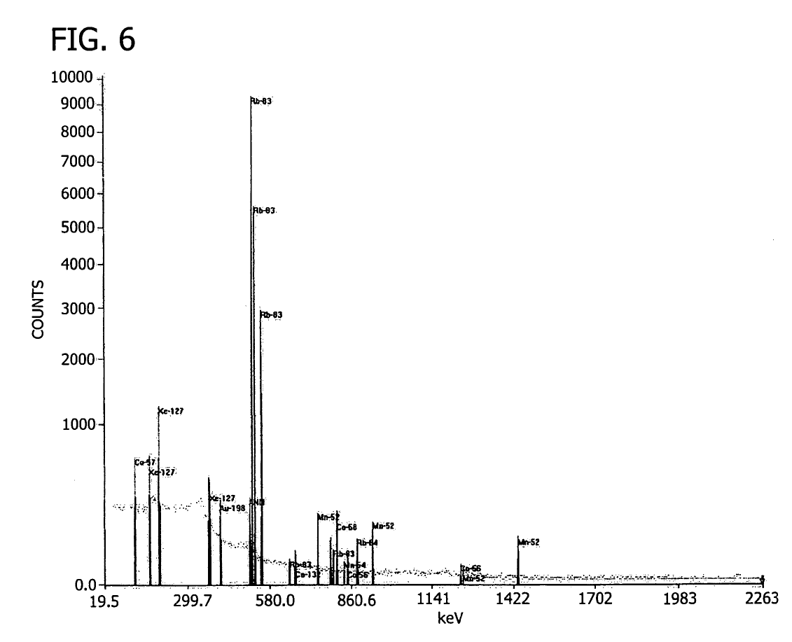

FIG. 6 shows a spectrum of Fraction 1 of generator 1 on Oct. 10, 2005.

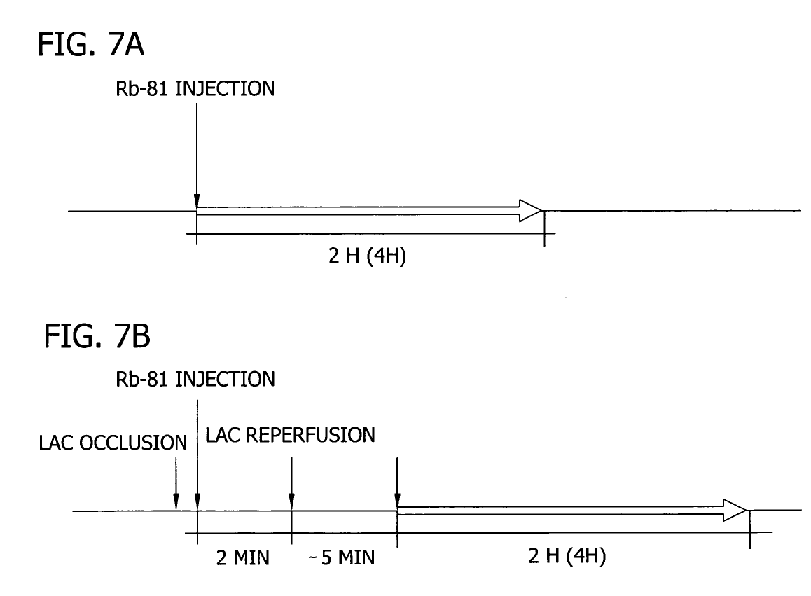

FIG. 7 shows imaging protocols for A) normal rats; and B) animals scheduled according the occlusion/reperfusion protocol used in Example 2.

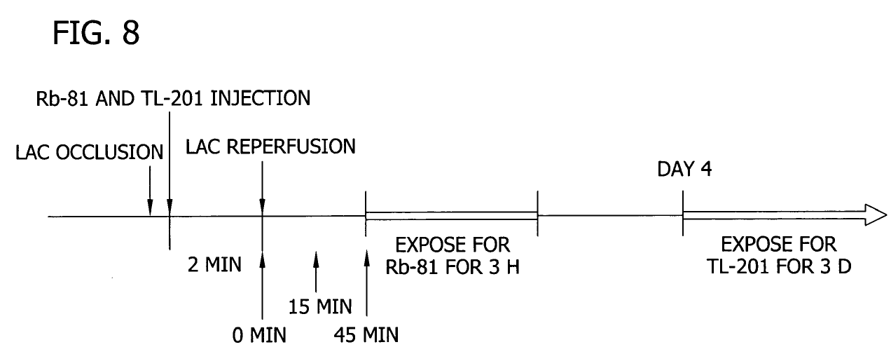

FIG. 8 shows protocols for ex-vivo autoradiography used in Example 2.

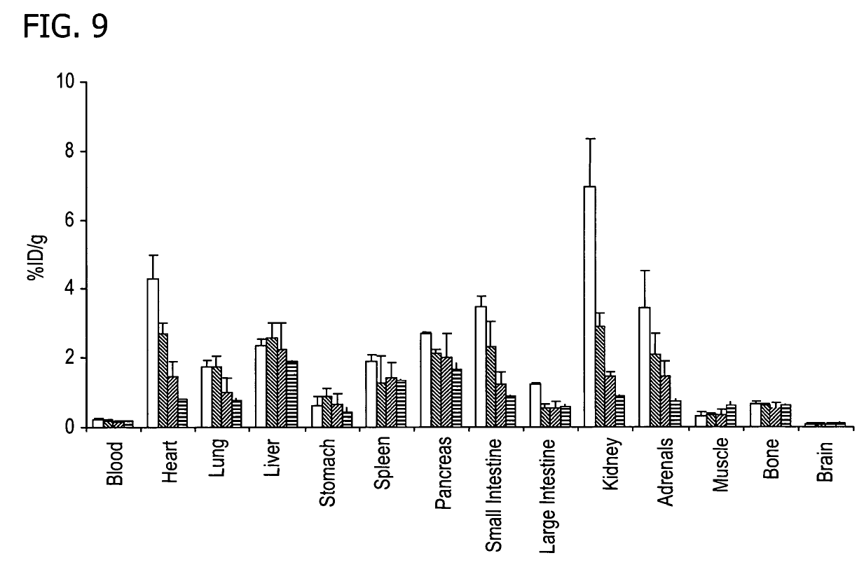

FIG. 9 shows the biodistribution in female Wistar rats at 10 min (left hand bars), 30 min (second bars from the left), 60 min (third bars from the left) and 4 h (right hand bars) after application of 131-144 μCi (4.85-5.33 MBq) of Rb-81 into the tail vein (n=3 animals for every time point).

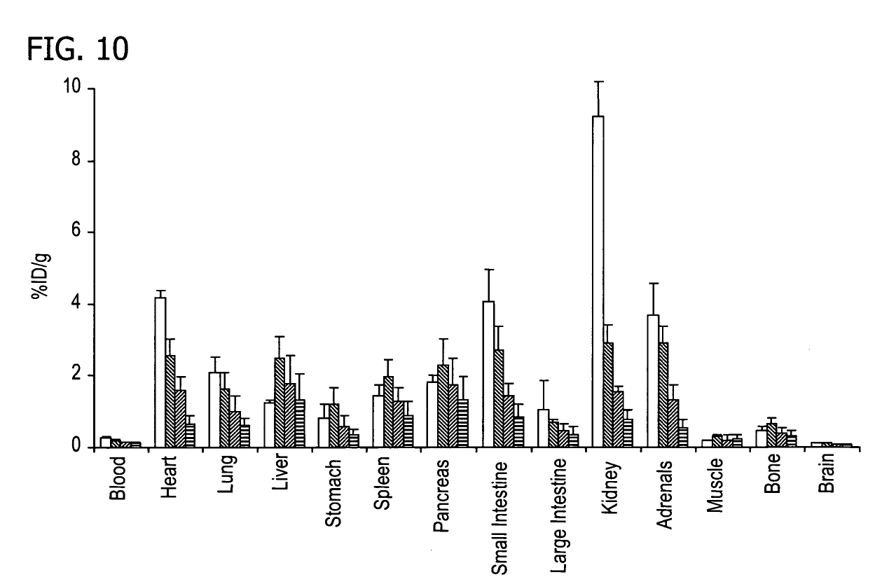

FIG. 10 shows the biodistribution in female Wistar rats at 5 min (left hand bars), 20 min (second bars from the left), 45 min (third bars from the left) and 2 h (right hand bars) after application of 50-137 μCi (1.85-5.87 MBq) of Rb-81 into the tail vein (n=3 animals for every time point).

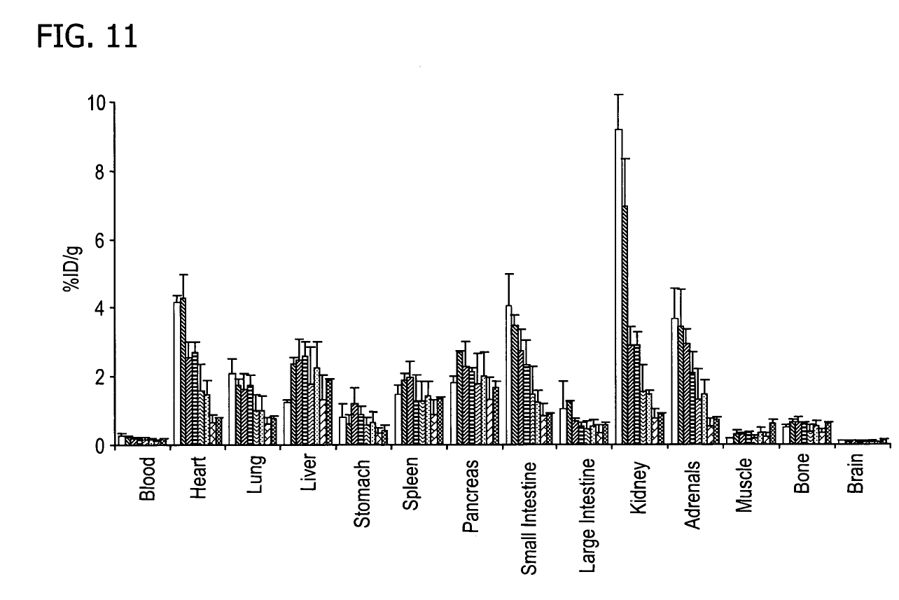

FIG. 11 shows a summary of the biodistribution experiments in female Wistar rats at 5 min, 10 min, 20 min, 30 min, 45 min, 1 h, 2 h, 4 h (from left to right) after application 1.85-5.87 MBq of Rb-81 into the tail vein (n=3 animals for every time point).

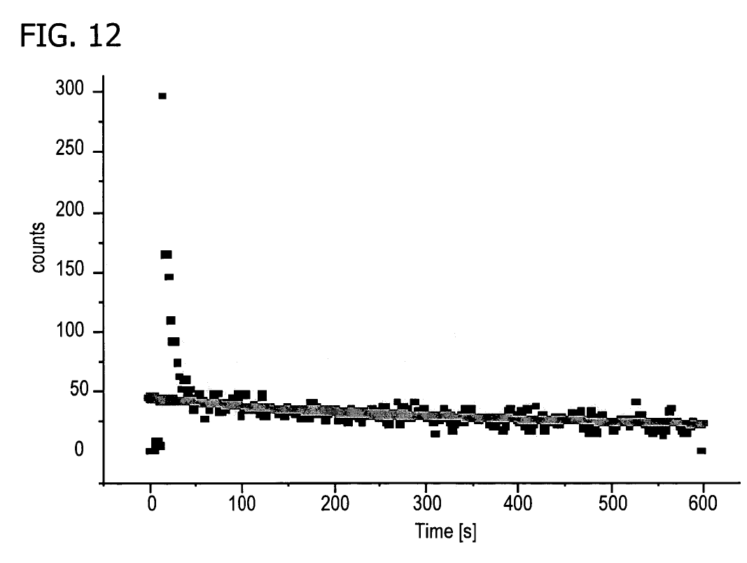

FIG. 12 shows transversal PET summation images of animal S2#3.

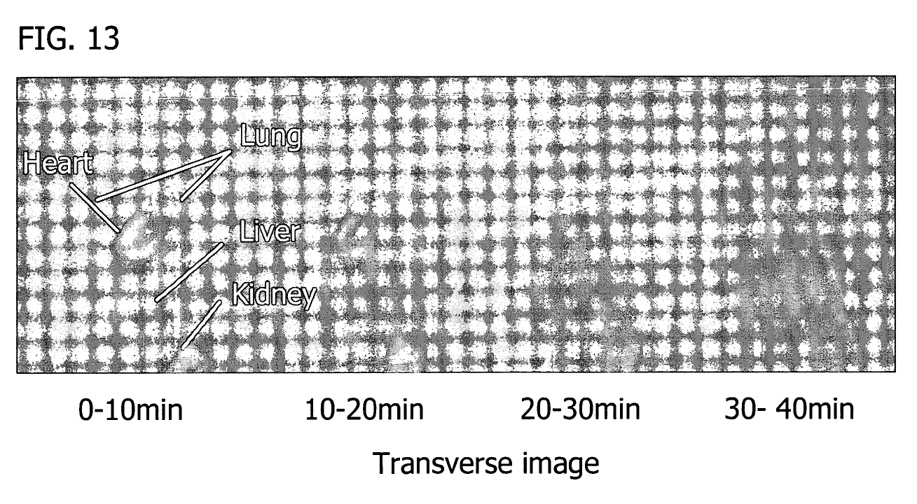

FIG. 13 shows short axis summation images of the heart of animal S2#3.

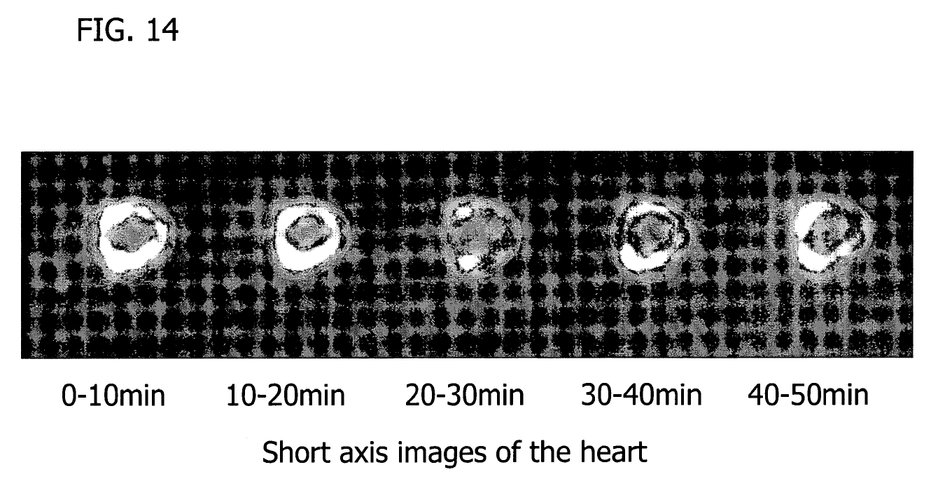

FIG. 14 shows short axis summation images of the heart of animal after occlusion/reperfusion (animal S2#4, for details see section Materials and Methods).

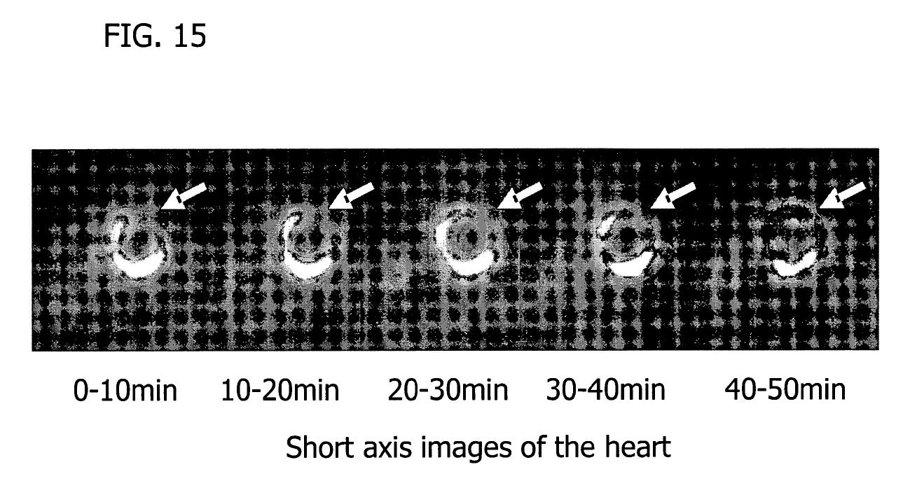

FIG. 15 shows autoradiographies of 20 μm heart slices prepared directly after initiating the reperfusion as well as 15 and 45 min after start of reperfusion.

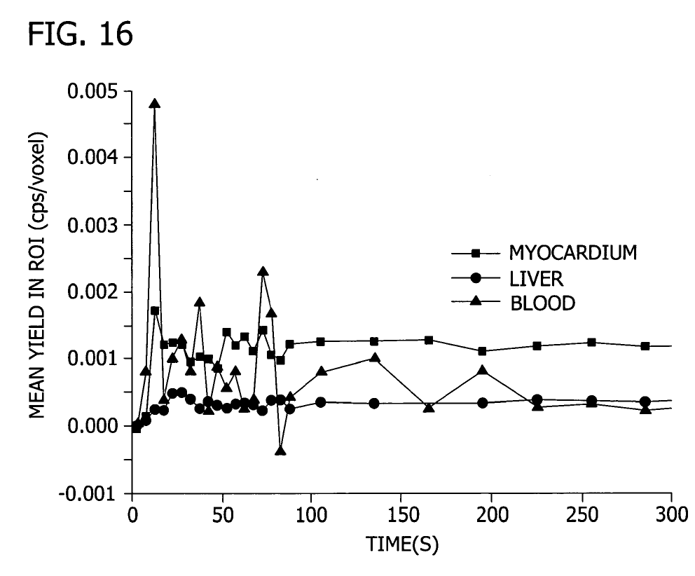

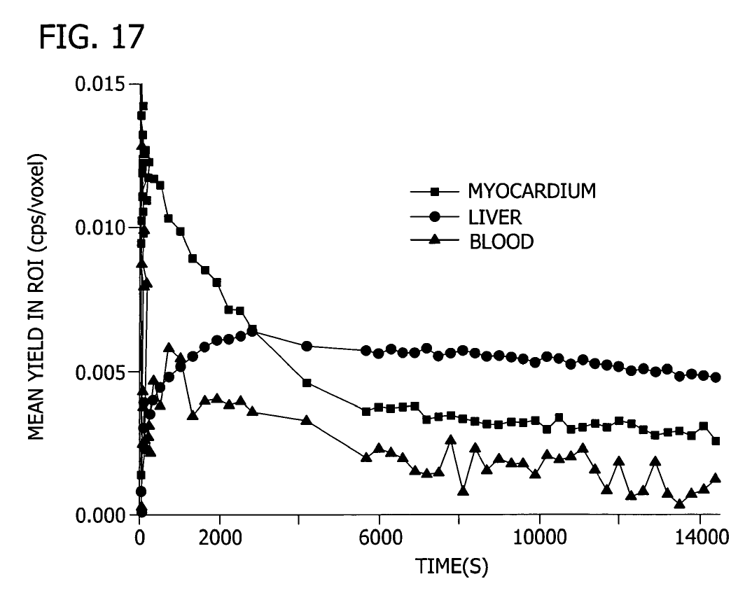

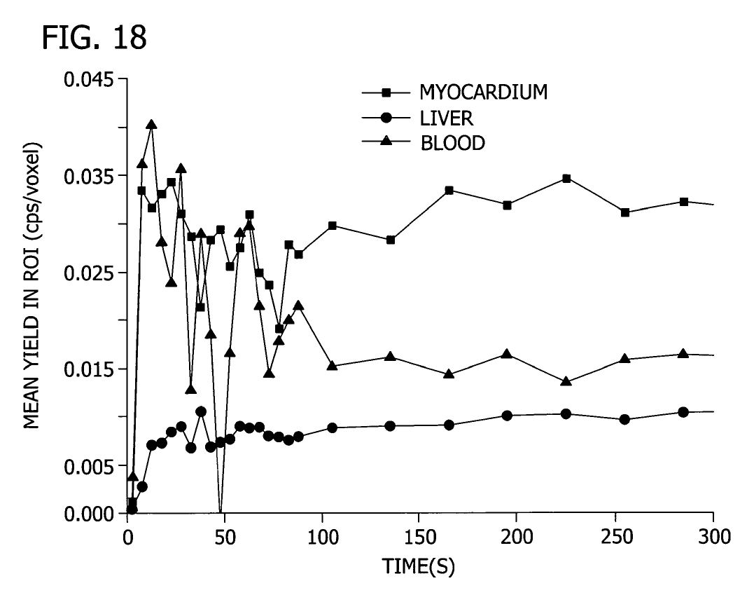

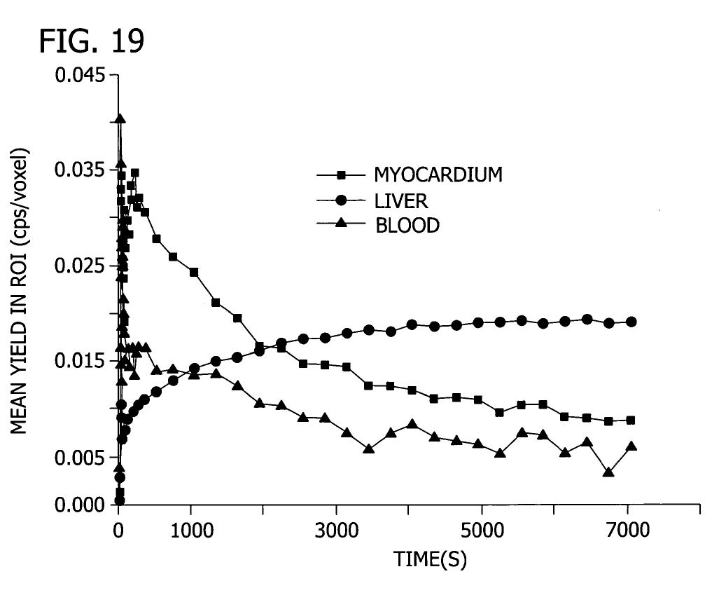

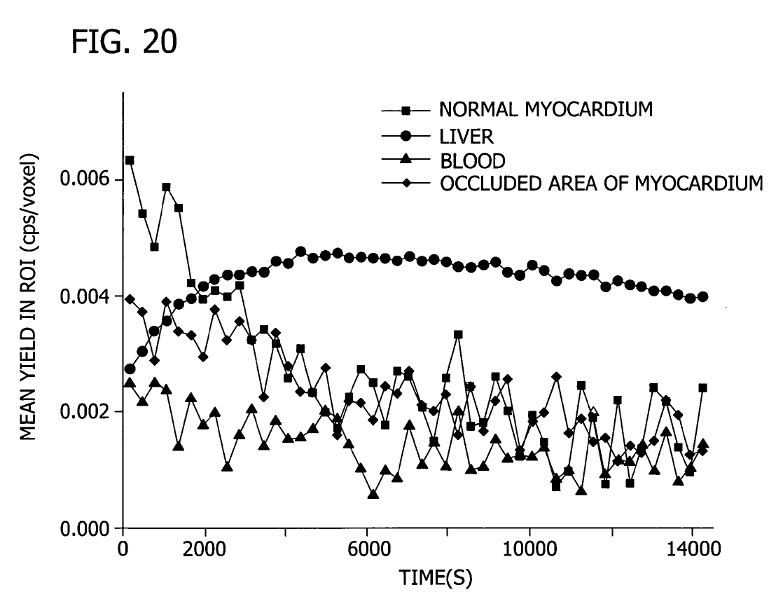

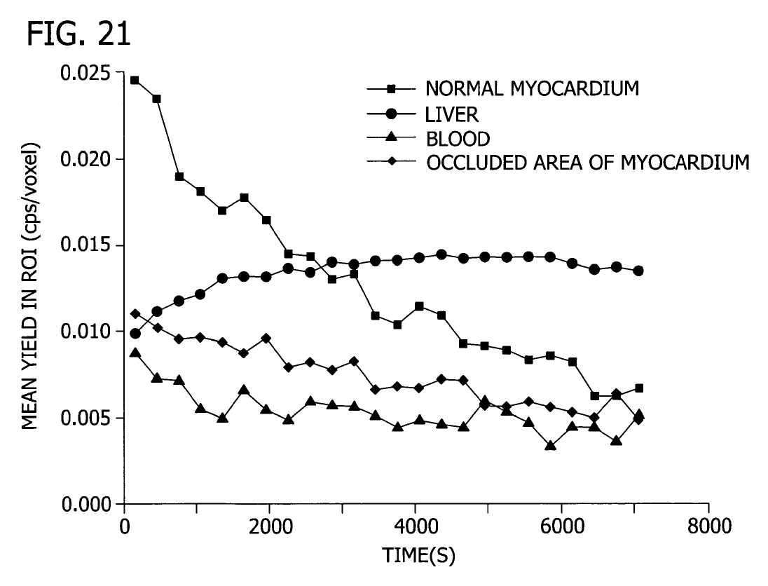

FIG. 16 shows quantitative comparison of Rb-81 and Tl-201 uptake in occluded versus normal heart tissue, expressed as percent of uptake in normal heart tissue.

FIG. 1 shows schematically a radioisotope generator (21) containing a cation-exchange material comprising resin beads (24), onto which are loaded the radioisotope Rb-81. When a cationic solution, for example saline, is introduced into the generator as shown by first arrow (22), the Rb-81 is eluted from the resin beads and an aqueous solution containing Rb-81 leaves the generator as shown by second arrow (23). If necessary, that solution may be diluted with a pharmaceutically-acceptable material such as further saline, sterilised for example by autoclaving, and then administered to a patient by intravenous injection prior to cardiac imaging, for example by a PET or SPECT procedure.

FIG. 2 shows a commercially-available KryptoScan™ generator, both in plan view and in side elevation, internal components being shown by dotted lines. Because of the hazardous radioactive material it contains, the generator is shielded with lead (not shown).

As shown in more detail in FIG. 3, the generator includes a membrane support (3) on which is located a cationic exchange membrane (4), both held within a housing (2). The housing (2) has an inlet side (13) and an outlet side (14), two tubes (10a, 10b) of plastics material provided with openable and closeable valves (7a, 7b respectively) enter the housing (2) at the inlet side (13) from the left-hand side as shown in FIG. 2. Another similar tube (12) leads to the inlet side (13) of the housing (2) but from the right hand side as shown in FIG. 2.

A further similar tube (11) enters the housing (2) from the right hand side as shown in FIG. 2, but communicates with the outlet side (14). Tubes (11) and (12) are also provided with openable and closeable valves (15) and (16) respectively.

The KryptoScan™ generator illustrated in FIGS. 2 and 3 may be used both in the preparation of Kr-81m gas for pulmonary imaging and for the generation of an Rb-81-containing solution for cardiac imaging.

When used for generating Kr-81m gas, the tubes (11) and (12) are simply used for loading the cationic exchange membrane (4) with Rb-81. In order to accomplish this Rb-81 solution is introduced into the housing (2) via tube (12) until such solution begins to leave the housing (2) by tube (11) whereupon loading is complete. The housing (2) is then heated to dry the membrane (4).

Kr-81m gas is generated by introducing air through one of the tubes (10a) or (10b) while the valves (7a) and (7b) are open and (15) and (16) are closed. This air becomes charged with Kr-81m (a decay product of Rb-81) as it passes over the membrane (4) and then leaves the housing through the other tube (10b) or (10a).

Alternatively, in order to obtain an aqueous solution of Rb-81, the valves (7a) and (7b) are closed and valves (15) and (16) opened. A physiologically-acceptable cationic solution (such as saline) is introduced into the inlet side (13) of the housing (2) via tube (12) so that it passes through the membrane (4) whereupon it elutes Rb-81 from the membrane and the resulting eluate solution containing Rb-81 leaves the outlet side (14) of the housing (2) through tube (11). If the concentration of Rb-81 in the solution is too high to permit it to be administered to a patient it is diluted as necessary with a physiologically-acceptable diluent such as saline. Preferably, the physiologically-acceptable cationic material and the diluent are both saline. Once a solution having the desired concentration of Rb-81 has been obtained it is sterilised by autoclaving whereupon it is ready for administration by intravenous injection to a patient who is to undergo cardiac imaging.

An important feature of the invention is that the same KryptoScan™ generator may first be used to obtain Kr-81m gas and then used to obtain a cationic solution containing Rb-81.

The Kr-81m is obtained by closing valves (15) and (16), opening valves (7a) and (7b), and passing air through the apparatus as described above.

Provided of course that sufficient undecayed Rb-81 remains after production of Kr-81m has been completed, the same generator may then be used to obtain a solution containing Rb-81 by closing valves (7a) and (7b) and opening valves (15) and (16), introducing the cationic solution into the housing via tube (12) and recovering the cationic solution containing Rb-81 via tube (11).

This is an important advantage of the invention as a single item of apparatus (e.g. the KryptoScan™ generator) can be used successively for two different procedures before being returned to the supplier to be reloaded with membrane charged with Rb-81.

The invention will now be further described with reference to the following Examples, which are intended to be purely illustrative only, and in no way intended to limit the scope of the invention.

### EXAMPLE 1

### Elution of Rb/Kr Generators with Physiological Salt

Three ⁸¹Rb-⁸¹mKr generators with a nominal value of 185 MBq loaded with ⁸¹Rb were eluted with 5×1 ml physiological salt. Under low vacuum, elution was performed with a pump speed of 5 ml/min.

The following elution profile of 81Rb was determined using a gamma radiation dose calibrator:

| | | Fraction | Fraction | Fraction | Fraction | Generator |
|---|---|---|---|---|---|---|
| | Fraction 1 | 2 | 3 | 4 | 5 | remainder |
| Generator 1 | 87% (154 MBq)^{a} | 7% | 2% | 0.4% | 0.2% | 4% |
| Generator 2 | 75% (111 MBq)^{a} | 14% | 4% | 1.3% | 0.7% | 5% |
| Generator 3 | 78% (120 MBq)^{a} | 11% | 3% | 1.3% | 0.7% | 5% |

^{a}From Ge/Li data of Oct. 6 and 7, 2005, error = +/−10%

The isotope composition of the eluate was on calibration time:

| Nuclide | Relative activity |
|---|---|
| ⁸¹Rb | 100 |
| ^{82m}Rb | 12.6 (+/−15%) |
| ⁸³Rb^{b} | 0.008 |
| ⁸⁴Rb^{c} | 0.0001 |
| ⁵²Mn^{c} | 0.0003 |

^{b}Data of Oct. 6, 7 and 10, 2005
^{c}Data of Oct. 7 and 10, 2005

Example spectra are shown in FIGS. 4-6.

### EXAMPLE 2

An animal study with Rb-81 has been carried out to demonstrate:
1) the biodistribution of Rb-81 in rats, i.e. the uptake kinetics and retention of Rb-81 in a rat heart;
2) the imaging of rat hearts using a small animal PET scanner;
3) the uptake kinetics of Rb-81 in an occlusion/reperfusion rat heart model in vivo; and
4) the comparison of the Tl-201 and Rb-81 redistribution kinetics in an occlusion/reperfusion rat heart model ex vivo.

### Materials and Methods

### a) Rb-81

Rb-81, eluted from Kr-81/Rb-81 generator with isotonic saline, was obtained and used without further purification or sterile filtration.

### b) Biodistribution of Rb-81 in Rats

1.85-5.33 MBq (50-144 μCi) Rb-81 in 250 μl isotonic saline was injected into the lateral tail vein of 24 non-fasted female Wistar rats weighing 180-220 g (12 animals at each day).

All the animals were anesthesized with Isofluran (1.5 vol. % and 2.01/min 02) during the injection of the Rb-81 solution. 5 min, 10 min, 20 min, 30 min, 45 min, 1 h, 2 h and 4 h post injection of the Rb-81 solution, the animals were sacrificed by carbon dioxide inhalation (n=3 animals for all time points).

The organs of interest (heart, blood, lungs, liver, stomach, spleen, pancreas, small intestine, colon, kidney, adrenal gland, muscle, bone, brain and tail) were dissected, weighed and measured for activity using a well-type g-counter. Tracer accumulation was normalized to the organ weights. All data are expressed as percent of the injected dose per gram tissue (% ID/g, mean±SD).

### c) Imaging of Rat Hearts Using a Small Animal PET Scanner

For the PET measurements, a microPET FOCUS 120 small animal scanner (Siemens Medical Solutions USA, Inc.) was used. Imaging was done on 4 female Wistar rats (200-250 g) (see Tab. 1). Two rats (one per day) had a left coronary artery occlusion (stress) during and for the following 5 min after the injection of 13.9 MBq Rb-81 and 36.1 MBq Rb-81, in 500 μl isotonic saline. After 5 min, the occlusion was removed and dynamic PET images of the animals were taken for 4 h and 2 h. Two rats were used as controls without occlusion (rest). The first rat was injected with 20.2 MBq Rb-81 and the second rat with 69.3 MBq Rb-81, in 500 μl isotonic saline. Following injection, dynamic PET images of the animals were taken for 4 h (S1#1 und S1#2) and 2 h (S2#3 and S2#4). The Rb-81 solution was injected intravenously in the lateral tail vein of all animals. For occlusion, a 7-0 polypropylene suture on a small curved needle was passed through the left coronary artery (LCA), and ligated to occlude the LCA. After tracer injection, reperfusion was obtained by cutting the suture. LCA occlusion and reperfusion were confirmed by the color change of myocardial surface.

Rats with an occlusion were anesthetized with a reversible triple anesthesia of 33.75 μg/kg Medetomidin, 0.45 mg/kg Midazolam and 1.125 u/kg Fentanyl during the occlusion and following injection of the Rb-81 solution. All four rats obtained Isofluran anesthesia (2.0 vol. % and 2.0 l/min O₂) during PET imaging.

After PET imaging, all animals were sacrificed by carbon dioxide inhalation and the organs of interest (see above) were dissected, weighed and measured for activity using a well-type g-counter. Tracer accumulation was normalized to the organ weights. All data are expressed as percent of the injected dose per gram tissue (% ID/g, mean±SD).

TABLE 1
μPET Imaging of Rats

| Animal No | Remarks | Injected Activity | Date |
|---|---|---|---|
| Rat S1#1 | Normal | 20.2 MBq | Jul. 20, 2006 |
| Rat S1#2 | 2 min occlusion/ | 13.9 MBq | Jul. 20, 2006 |
| | Reperfusion | | |
| Rat S2#3 | Normal | 69.3 MBq | Aug. 03, 2006 |
| Rat S2#4 | 2 min occlusion/ | 36.1 MBq | Aug. 03, 2006 |
| | Reperfusion | | |

### d) Data Acquisition

The normal rats were anesthetized and positioned in the scanner. A point source based transmission measurement was followed by on bed injection of the Rb-81. Animals following the occlusion-protocol were positioned in the scanner after initiation of the reperfusion. Subsequently, the measurement was started (entailing a switch to isoflurane anesthesia). Imaging protocols are shown in FIG. 7.

### e) Ex Vivo Dual Tracer Autoradiography

Three additional rats were simultaneously injected with 20 MBq Rb-81 and 2.7 MBq Tl-201. After the occlusion/injection/reperfusion procedure was completed (see FIG. 8), the animals were sacrificed at 0, 15 and 45 min after initiating the reperfusion.

After the hearts of the animals were removed and freshly frozen in liquid nitrogen, they were sliced into short axis 20 μm slices. Radioactivity in the tissues sections was determined by using a phosphor-imager. Rb-81 was measured over a period of 3 h directly after preparation of the slices, whereas the Tl-201 activity was determined after the Rb-81 activity was almost completed (Rb-81: t1/2=4.58 h versus a 4 day decay period).

Quantitative evaluations were carried out by comparison of the detected counts within regions-of-interest in both, normal tissue areas and occluded tissues areas. Comparisons are given in percent of normal heart tissue.

### Results

### 1. Biodistribution of Rb-81 in Rats

Reference is made to FIGS. 9-11 which show the biodistribution in female Wistar rats at various time intervals after application of 131-144 μCi (4.85-5.33 MBq) of Rb-81 into the tail vein (n=3 animals for every time point).

Table 2 shows a summary of the biodistribution experiments in female Wistar rats at 5 min, 10 min, 20 min and 30 min after application of Rb-81 into the tail vein (n=3 animals for every time point).

TABLE 2
Summary of the biodistribution experiments in female Wistar rats
at 5 min, 10 min, 20 min and 30 min after application of Rb-81
into the tail vein (n = 3 animals for every time point).

| Organs | 5 min | 10 min | 20 min | 30 min |
|---|---|---|---|---|
| Blood | 0.25 ± 0.07 | 0.22 ± 0.04 | 0.21 ± 0.04 | 0.19 ± 0.03 |
| Heart | 4.18 ± 0.18 | 4.30 ± 0.68 | 2.55 ± 0.47 | 2.69 ± 0.32 |
| Lung | 2.09 ± 0.43 | 1.74 ± 0.18 | 1.62 ± 0.46 | 1.73 ± 0.31 |
| Liver | 1.25 ± 0.05 | 2.37 ± 0.20 | 2.47 ± 0.64 | 2.60 ± 0.43 |
| Stomach | 0.80 ± 0.41 | 0.63 ± 0.26 | 1.20 ± 0.46 | 0.88 ± 0.24 |
| Spleen | 1.45 ± 0.30 | 1.88 ± 0.21 | 1.97 ± 0.47 | 1.26 ± 0.78 |
| Pancreas | 1.81 ± 0.20 | 2.70 ± 0.02 | 2.29 ± 0.73 | 2.12 ± 0.13 |
| Small Intestine | 4.06 ± 0.91 | 3.47 ± 0.33 | 2.73 ± 0.64 | 2.33 ± 0.72 |
| Large Intestine | 1.03 ± 0.82 | 1.22 ± 0.05 | 0.70 ± 0.07 | 0.53 ± 0.14 |
| Kidney | 9.21 ± 0.98 | 6.96 ± 1.39 | 2.90 ± 0.51 | 2.91 ± 0.37 |
| Adrenals | 3.68 ± 0.89 | 3.44 ± 1.07 | 2.92 ± 0.45 | 2.08 ± 0.60 |
| Muscle | 0.18 ± 0.02 | 0.31 ± 0.12 | 0.31 ± 0.02 | 0.36 ± 002 |
| Bone | 0.48 ± 0.10 | 0.66 ± 0.09 | 0.65 ± 0.15 | 0.63 ± 0.03 |
| Brain | 0.10 ± 0.02 | 0.09 ± 0.03 | 0.11 ± 0.02 | 0.09 ± 0.01 |

Table 3 shows a summary of the biodistribution experiments in female Wistar rats at 45 min, 1 h, 2 h and 4 h after application of Rb-81 into the tail vein (n=3 animals for every time point).

TABLE 3
Summary of the biodistribution experiments in female Wistar rats
at 45 min, 1 h, 2 h and 4 h after application of Rb-81 into the
tail vein (n = 3 animals for every time point).

| Organs | 45 min | 60 min | 120 min | 240 min |
|---|---|---|---|---|
| Blood | 0.15 ± 0.08 | 0.17 ± 0.01 | 0.10 ± 0.05 | 0.20 ± 0.01 |
| Heart | 1.58 ± 0.79 | 1.48 ± 0.41 | 0.67 ± 0.21 | 0.81 ± 0.02 |
| Lung | 1.02 ± 0.45 | 1.00 ± 0.44 | 0.60 ± 0.23 | 0.79 ± 0.05 |
| Liver | 1.79 ± 1.07 | 2.25 ± 0.77 | 1.32 ± 0.73 | 1.90 ± 0.03 |
| Stomach | 0.57 ± 0.24 | 0.64 ± 0.31 | 0.34 ± 0.15 | 0.44 ± 0.15 |
| Spleen | 1.27 ± 0.57 | 1.43 ± 0.41 | 0.88 ± 0.42 | 1.34 ± 0.06 |
| Pancreas | 1.76 ± 0.91 | 1.99 ± 0.73 | 1.31 ± 0.66 | 1.65 ± 0.20 |
| Small Intestine | 1.45 ± 0.82 | 1.25 ± 0.34 | 0.83 ± 0.37 | 0.89 ± 0.02 |
| Large Intestine | 0.47 ± 0.22 | 0.53 ± 0.20 | 0.36 ± 0.24 | 0.59 ± 0.05 |
| Kidney | 1.56 ± 0.77 | 1.45 ± 0.14 | 0.76 ± 0.27 | 0.90 ± 0.04 |
| Adrenals | 1.32 ± 0.88 | 1.45 ± 0.43 | 0.56 ± 0.22 | 0.75 ± 0.07 |
| Muscle | 0.21 ± 0.06 | 0.35 ± 0.14 | 0.24 ± 0.13 | 0.61 ± 0.11 |
| Bone | 0.38 ± 0.18 | 0.54 ± 0.16 | 0.33 ± 0.12 | 0.61 ± 0.04 |
| Brain | 0.07 ± 0.03 | 0.08 ± 0.02 | 0.06 ± 0.03 | 0.13 ± 0.01 |

### 2. Imaging of Rat Hearts Using a Small Animal PET Scanner

For the PET measurements a microPET FOCUS 120 small animal scanner (Siemens Medical Solutions USA, Inc.) was used. Imaging was done with 4 female Wistar rats (200-250 g) (see Table 1). Images are shown in FIGS. 13-15.

FIG. 13 shows that uptake of 81-RB in the myocardium is highest shortly after administration.

FIG. 14 shows uptake in the normal myocardium, over time.

FIG. 15 shows an animal where a temporary occlusion of the LAC has been performed. Over a period of 40-50 minutes after re-opening of the occlusion, filling in of the defect takes place (redistribution of 81-RB).

### 3. Comparison of the Tl-201 and Rb-81 Redistribution Kinetics in an Occlusion/Reperfusion Rat Heart Model Ex Vivo

Assuming an almost complete decay of the Rb-81 activity (20 MBq at the time point of injection) 4 d after the first measurement, the residual Tl-201 activity (2.7 MBq at the time point of injection) on the tissue slices should represent the reperfusion of this isotope at 0 min, 15 min and 45 min post injection. In contrast, the early images from the phosphor-imager screen should represent predominantly the Rb-81 activity distribution.

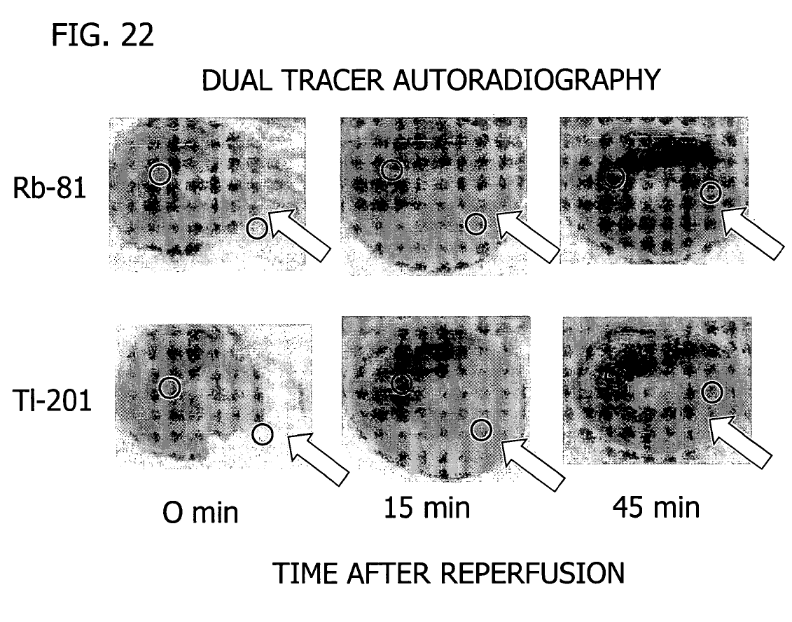

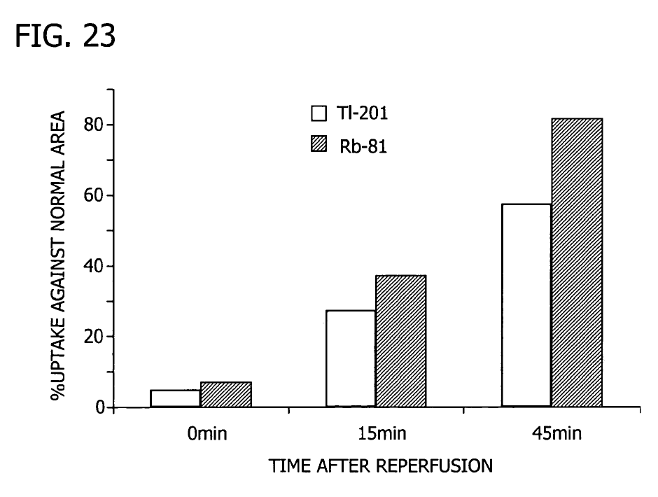

As shown in FIG. 22, occluded areas can be clearly delineated at 0 min and 15 min p.i., whereas the late image at 45 min demonstrates an advanced redistribution of Rb-81. Although the redistribution of Tl-201 has already reached a significant level at the same time point, Rb-81 redistribution kinetics has been found to be considerably faster.

Quantitative relations were obtained by determination of the activity in regions of interest (ROIs) placed in the normal and occluded areas of the hearts. The quantitative data revealed, that Tl-201 activity in occluded areas reached 5%, 28% and 58% of the uptake in normal tissues at 0 min, 15 min and 45 min p.i., respectively. In contrast, the uptake of Rb-81 was somewhat higher in occluded areas, thus demonstrating faster redistribution (7%, 37% and 82% at 0 min, 15 min and 45 min p.i., respectively).

In conclusion, Rb-81 is a unique PET tracer which can assess tracer accumulation over a long time period which enables the assessment of the viability of myocardium with stress and late redistribution protocols in patients with coronary artery disease using PET.

1. A method of preparing an imaging composition containing Rb-81 substantially free from radioactive non-Rb impurities, the method comprising:
washing a cation-exchange material with water, to which cation-exchange material is bound Rb-81, to remove radioactive non-Rb impurities and then eluting said cation-exchange mixture with a pharmaceutically-acceptable cationic solution, wherein the eluting releases Rb-81 from the cation-exchange material.

2. A method according to claim 1 wherein the pharmaceutically-acceptable cationic solution is isotonic saline solution.

3. A method according to claim 1, wherein the cation-exchange material comprises resin beads, membrane including beads, or membrane.

4. A method according to claim 3, wherein the cation-exchange material comprises a polymer of at least one of divinylbenzene/styrene and ethylvinylbenzene.

5. An imaging composition comprising:
a pharmaceutically-acceptable solution containing Rb-81 substantially free from non-Rb radioactive impurities in a pharmaceutically-acceptable cationic solution.

6. A composition comprising Rb-81 retained on a cation-exchange material.

7. A composition according to claim 6, wherein the cation-exchange material comprises resin beads, membrane including beads, or membrane.

8. A composition according to claim 7, wherein the cation-exchange material comprises a polymer of at least one of divinylbenzene/styrene and ethylvinylbenzene.

9. A method for the preparation of an imaging composition including a pharmaceutically-acceptable solution of Rb-81, the method comprising:
(i) introducing enriched Kr-82 gas into a target in a cyclotron;
(ii) irradiating said enriched Kr-82 gas target within the cyclotron so as to generate radioactive Rb isotopes and radioactive non-Rb impurities;
(iii) washing the target with water to obtain an Rb-81-containing aqueous solution containing said impurities;
(iv) loading the resulting radioactive Rb containing aqueous solution onto a cation-exchange resin;
(v) washing said cationic-exchange resin with water to remove said impurities;
(vi) eluting the cationic-exchange resin with a physiologically-acceptable cationic solution so as to dissociate radioactive Rb from the resin;
(vii) collecting the resulting purified aqueous Rb solution;
(viii) sterilising the resulting solution.

10. A method according to claim 9, wherein after step (vii) and prior to step (viii) the Rb-81-containing solution is diluted with a pharmaceutically-acceptable diluent.

11. A method according to claim 10, wherein said diluent is a further quantity of said physiologically-acceptable cationic solution.

12. A method according to claim 9, wherein the pharmaceutically-acceptable cationic solution is isotonic saline solution.

13. Use in diagnostic imaging by PET or SPECT of an imaging composition comprising a pharmaceutically-acceptable cationic solution containing Rb-81 substantially free from radioactive impurities.

14. Use according to claim 13, wherein the imaging is of cardiac tissue.

15. Use according to claim 14, wherein the cardiac tissue is myocardial tissue.

16. Use according to claim 13, where the imaging is of potassium channels in cells.

17. A method of performing a radioisotope elution procedure, the method comprising:
(i) eluting a cationic-exchange resin having radioactive Rb-81 thereon with a physiologically-acceptable gas so as to provide a gas mixture including the gas and Kr-81m formed by decay of the radioactive Rb-81; and
(ii) subsequently eluting the cationic-exchange resin still having at least some of the radioactive Rb-81 thereon with a physiologically-acceptable cationic solution so as to dissociate the radioactive Rb-81 from the resin.

18. A method according to claim 17, wherein the physiologically-acceptable gas is air or oxygen.

19. A method according to claim 17, wherein the physiologically-acceptable cationic solution is an isotonic saline solution.

20. (canceled)
